(19) United States
(12) Patent Application Publication
(10) Pub. No.: US 2008/0176248 A1
(43) Pub. Date: Jul. 24, 2008

(54) Phage display affinity and specificity filters and forward screen

(75) Inventors: David J. Lockhart, Del Mar, CA (US); Daniel Kelly Treiber, San Diego, CA (US); Patrick Parvis Zarrinkar, San Diego, CA (US)

Correspondence Address: JONES DAY, 222 EAST 41ST ST, NEW YORK, NY, 10017, US

(73) Assignee: Ambit Biosciences Corp., San Diego, CA (US)

(21) Appl. No.: 11/982,389

(22) Filed: Oct. 31, 2007

Related U.S. Application Data
(63) Continuation of application No. 10/406,797, filed on Apr. 2, 2003, now abandoned.
Application No. 10/406,797 is a continuation-in-part of application No. 10/115,442, filed on Apr. 2, 2002, now abandoned.

(51) Int. Cl. G01N 33/53

(52) U.S. Cl. 435/7.2

(57) ABSTRACT

The invention provides an affinity filter for the binding of phage-displayed proteins to dissolved target molecules. The phage-displayed proteins are contacted with immobilized target in the presence and absence of dissolved target; the behavior of the phage-displayed proteins as a function of concentration of dissolved target permits approximation of the affinity of the phage-displayed protein for target. The invention also provides a method to screen large numbers of compounds for their ability to compete with a compound known to bind a phage-displayed protein.

### CROSS-REFERENCE TO RELATED APPLICATIONS

This application is a continuation of U.S. Ser. No. 10/406,797, filed Apr. 2, 2003, which is a continuation-in-part of U.S. Ser. No. 10/115,442 filed Apr. 2, 2002 (abandoned). The contents of these applications are incorporated herein by reference.

### TECHNICAL FIELD

The invention relates to the study of interactions of molecules where phage display is employed to provide a library of proteins or peptides. More particularly, it relates to methods to define and validate the interaction of phage-displayed proteins with their targets in terms of the strength of this interaction. The invention also includes methods to employ similar techniques to discover alternative molecules which bind to a target protein in a “forward screen,” and to assure binding specificity in a “specificity filter.”

### BACKGROUND ART

Methods to display a wide variety of peptides or proteins as fusions with coat protein of bacteriophage is well known. The original system was disclosed, for example, in U.S. Pat. Nos. 5,096,815 and 5,198,346. This system used the filamentous phage M13 which required that the cloned protein be generated in E. coli and required translocation of the cloned protein across the E. coli inner membrane. Lytic bacteriophage vectors, such as lambda, T4 and T7 are more practical since they are independent of E. coli secretion. T7 is commercially available and described in U.S. Pat. Nos. 5,223,409; 5,403,484; 5,571,698 and 5,766,905.

Traditionally, the phage display system has been used to examine the interaction of the phage-displayed proteins with proteins or peptides. An initial important application of phage display, for example, was the production of single chain antibody variable regions which could then be tested for interaction with a specific antigen. The system could be used to develop specific antibodies for a particular antigen.

More recently, it has been found possible to use phage display techniques to explore interactions between proteins or peptides and “small molecules”—i.e., typically synthetic organic molecules which may be useful as pharmaceutical compounds. This technique is described in PCT publication WO01/18234 published 15 Mar. 2001. In one embodiment of this application, the biological targets for known pharmaceuticals can be ascertained by displaying the protein products of cDNA libraries and using a known pharmaceutical as a target.

Regardless of the application of the phage display technique, there is a problem of “false positives,” and, in addition, when repeated rounds of selection are used, the highest affinity binders may obscure the effect of important, but weaker, interactions. The present invention overcomes these problems in the phage display technology by supplying an “affinity filter” as described below.

Using “affinity filters” in the form of competitive binding is known in other contexts. Competitive binding to establish dissociation constants is a standard laboratory technique. For example, a very early paper by Lin, S-Y, et al., J. Mol. Biol. (1972) 72:671-690 describes competition experiments for measuring the interaction of E. coli lac repressor with various DNA compositions. More recently, Knockaert, M., et al., Chem. & Biol. (2000) 7:411-422 describe a competition reaction between varying amounts of ATP in brain extracts in testing their interaction with a matrix supporting oocyte extracts to assess the degree of binding of factors in the brain extracts, (CDK5 and ERK1/2) for binding to the oocyte components. However, to applicant's knowledge, competition assessment to behave as an affinity filter has not been applied to phage display, except to the limited extent disclosed by Danner, S., et al., Proc. Natl. Acad. Sci. USA (2001) 98:12954-12959. In this paper, it was shown that use of competitor RNA could improve detection of a phage-displayed protein that is known to bind RNA more weakly than a second phage-displayed protein also present in the phage lysate. No detection of false positives was reported.

Similarly, use of competitive binding to determine the ability of a compound to bind, for instance, to a receptor, is widely employed. For example, the ability of a compound A to bind to receptor B is frequently ascertained by measuring the ability of compound A to displace a labeled known binder from the receptor. Again, however, to applicant's knowledge this technique has not been employed as a forward screen for additional molecules which bind a phage-displayed protein once an initial binder for the phage-displayed protein has been found. A single instance of employing competitive binding in relation to specificity has been reported by Dennis, M. S. et al., J. Biol. Chem. (1994) 269:22137-22144. In this paper, Kunitz domain mutants were selected that bind to Factor VIIa:TF but not to Factor IXa. A pool of mutant Kunitz domains was displayed on phage and the Factor VIIa:TF was immobilized to solid support; soluble Factor IXa was included as a competitor to eliminate retention of phage that bind to both targets.

### DISCLOSURE OF THE INVENTION

The invention relates to a technique to assess the affinity of the interaction of a target moiety with phage-displayed proteins or peptides. The system obviates the problem of false positives, permits discovery of interactions of only moderate affinity, and allows an estimation of affinity constants for the target/displayed protein interaction. The invention also provides a method to screen for a multiplicity of alternative compounds which could substitute for compounds that have been found to bind to a particular phage-displayed protein, and to assure specificity of binding.

Thus, in one aspect, the invention is directed to an improved method to perform phage display analysis that can be considered an affinity filter. Said analysis comprises the step of detecting the binding of a phage-displayed protein to a target coupled to a solid support, wherein the improvement comprises including, in at least one sample of the fluid phase which contains the phage-displayed proteins, a concentration of the target sufficient to diminish the binding of the displayed protein to the solid support.

In more detail, phage display is performed in a conventional manner except that the phage bound to target immobilized on solid support are recovered, amplified and detected in the absence of dissolved target, in the presence of low concentrations of dissolved target and/or in the presence of high concentrations of dissolved target. Phage which remain bound to the immobilized target under all three conditions (or the first and third) are identified as non-specific false positives. Phage which remain bound to immobilized target only at low concentrations of dissolved target but not at high concentrations of dissolved target are identified as moderately binding proteins. Phage-displayed proteins which are detected bound to immobilized target in the absence of dissolved target, but which are no longer detectable even at low concentrations of dissolved target are identified as high affinity binders.

By use of this method of the invention, false positives are identified, and moderate binders which would otherwise have escaped detection can be shown to be present. Thus, in other aspects, the invention is directed to methods to identify false positive binders, and methods to discover moderate binding of phage-displayed proteins which would otherwise have escaped detection.

In another aspect, the invention is directed to a method to determine the dissociation constant of a target and the phage-displayed protein to which it binds which method comprises assessing the binding of the displayed protein to immobilized target in the presence of various concentrations of target in solution, whereby an approximation of the dissociation constant is obtained by evaluating the concentration at which half of the phage-displayed protein is bound to the support and half is unbound.

In still another aspect, the invention is directed to a method to discover in a “forward screen” alternative compounds which bind the phage-displayed protein in addition to the target itself. Once the target molecule has been identified, additional compounds which bind the phage-displayed proteins can be efficiently discovered by evaluating the ability of dissolved or free candidate compounds to compete with immobilized target for binding the phage-displayed protein. This aspect takes advantage of the same principle as the affinity filter—i.e., a compound in solution which successfully binds the phage-displayed protein will displace phage from solid support which are bound to the identified target or parental molecule that has been immobilized. The concentration required to displace the phage from the immobilized target is also a measure of the strength of binding of the competitor. This “forward assay” can be made particularly efficient by first testing pools of candidates and testing individual candidates only from successful pools.

Thus, in this aspect, the invention is directed to a method to identify a compound that binds a phage-displayed protein which method comprises contacting a solid support containing an immobilized target molecule known to bind said phage-displayed protein with a lysate containing phage displaying said protein in the presence and absence of at least one candidate compound; titrating the amount of phage bound to the support in the presence and in the absence of said candidate compound and comparing the titers in each case, whereby a reduction in the titer of bound phage in the presence as opposed to the absence of the candidate compound identifies the candidate as able to bind the phage-displayed protein.

Still another aspect of the invention relates to a specificity filter, whereby phage-displayed proteins can be identified that bind to a specific target compound, but which do not bind to alternative compounds for which interaction is not desired. In this aspect, the first compound, for which a specifically interacting protein is desired, is immobilized on a solid support which is then treated with a fluid containing a multiplicity of displayed proteins and at least a second compound with respect to which protein binding is not desired. At sufficient concentrations of compound for which binding is not desired, any proteins which bind non-specifically—i.e., bind both to the desired target and to the additional alternative compounds, will be prevented from being retained on the solid support. Thus, only phage-displayed proteins that specifically bind the compound coupled to solid support will be retained; those which lack sufficient specificity will be filtered out.

### BRIEF DESCRIPTION OF THE DRAWINGS

FIG. 1 is a diagrammatic representation of results obtained in the method of the invention.

FIG. 2 is a photocopy of a gel displaying PCR amplified eluates of phage containing displayed FKbp proteins from a solid support on which FK506 is immobilized in the presence of varying amounts of rapamycin competitor.

FIG. 3 is a photocopy of a gel displaying PCR amplified eluates of phage containing displayed SHC PTB domains from a solid support on which trkA-PY490 peptide is immobilized in the presence of varying amounts of competitor.

FIG. 4 is a graph showing the effect of competitor concentration on protein bound to the solid support.

FIG. 5 is a graph showing additional examples of the effect of competitor concentration on protein bound to solid support.

FIG. 6 shows the results of high throughput forward screens using compound pooling.

FIG. 7 shows the ability of the invention method to reveal occult moderate binders.

FIG. 8 is a photocopy of a gel displaying PCR amplified eluates of phage containing displayed kinase proteins from four different solid supports to which various known kinase inhibitors are immobilized. The results shown are obtained in the absence and presence of either the free form of the immobilized compound (affinity filter) or free forms of alternative inhibitors (specificity filter).

### MODES OF CARRYING OUT THE INVENTION

The invention concerns interactions between a phage-displayed protein and a “target.” As used in the present application, “target” refers to the molecule (be it protein, small molecule, carbohydrate, or other embodiment) which the phage-displayed protein is to be tested for binding. The “immobilized target” refers to this molecule coupled to solid support; “target in solution” or “dissolved target” is then self-explanatory. Immobilization of the target is by a variety of means, and standard ways of coupling targets to solid supports are well known in the art, including the use of linker molecules, crosslinkers such as glutaraldehyde, biotin/avidin interactions, for example, between biotin coupled to the target and avidin bound to a solid support. The solid support itself can take any convenient form, typically a column containing particles to which the target is immobilized or a planar surface containing immobilized target.

The phage-displayed protein is produced as a fusion protein with a coat protein characterizing the phage. The displayed, non-phage protein can be coupled to the C-terminus or the N-terminus of the coat protein characteristic of the phage. In a preferred embodiment, the non-phage protein to be studied is coupled to the C-terminus of the coat protein in order to avoid instances wherein a stop codon contained in the non-phage protein interrupts translation before the nucleotide sequence encoding the coat protein is even reached.

As described in the documents referenced in the Background section hereinabove, and which are incorporated herein by reference, the traditional method of discovering partners in an interaction between a protein and an additional molecule (target) where the protein is provided through phage display is conducted as follows: a substance for which a protein partner is to be sought is coupled to a solid support. The solid support is then treated with a fluid containing a phage display library, under conditions wherein the displayed proteins will bind to immobilized target, ideally based on a specific interaction between one or more particular members of the library and the immobilized target. The phage display library is composed of a multiplicity of different proteins. This multiplicity may be generated by expression of nucleotide sequences subjected to random mutations, expression from systematically synthesized variants of DNA, expression from a cDNA library, or prepared by a variety of other means as long as a multiplicity of proteins is generated. The proteins in the library, by virtue of their being fused to a phage coat protein are displayed on the surface of the phage. Those phage-displayed proteins which interact with the “target” immobilized on the support are themselves bound to the support by virtue of their interaction with the target. The support is then washed, if necessary, to remove unbound phage, and the coupled phage is then eluted and characterized.

To characterize the eluted phage, the successfully bound phage can be amplified by infecting bacteria. The amplified phage are then analyzed. Either the displayed proteins can be extracted and characterized or the genome or a portion thereof of the amplified phage can be displayed. Typically, the amplified genome, nucleic acid fragment or amplified fusion coat protein is subjected to size separation on a gel; there is no need further to characterize the proteins or nucleic acids as the method of the invention will discriminate those phage which warrant further characterization from those which do not. Those proteins which are then identified as strong or moderate binders can be further characterized using standard techniques, for example, by sequencing the DNA which encodes them. Such further characterization is comparatively tedious and a distinct advantage of the invention is its ability to limit the necessity for characterization to those proteins displayed by phage which are truly able to bind the target in dissolved form.

The foregoing process can, if desired, be repeated for additional “rounds” of selection to isolate a homogeneous phage population. Commonly, a mixture of phage is bound to the target; more copies of phage displaying a protein with high affinity than copies of phage displaying protein with weaker affinity for the target are typically bound. In the amplification step, then, the concentration of the higher affinity binding phage is enhanced and in the subsequent rounds of selection, the balance is tipped even more strongly in favor of the high affinity protein. Multiple rounds of amplification thus may isolate a high affinity binder to the target to the exclusion of proteins with moderate but significant affinity. Such moderate binders, in the case of binding to a pharmaceutical, for example, may represent the potential for side effects of the pharmaceutical. Thus, one problem associated with this technique in general is the masking of moderate, but important, interactions between the target and a protein by interactions between the target and proteins that are more strongly bound.

The present invention solves the forgoing problem by permitting detection of these moderate binders. While any quantitative definition of high and moderate binders is clearly arbitrary, in many instances, but not necessarily all, high affinity binders would have K_{d} values of <1 μM and moderate binders would have K_{d}'s in the range of 1-100 μM. However, these ranges will vary depending on the nature of the interaction sought. The advantage of the method of the invention is that a comparatively moderate binders whose presence would otherwise be undetected when a high affinity binder is present can, using the invention methods, be discovered.

Another problem relates to false positives. A library which in fact does not contain any proteins which bind significantly specifically to the target may nevertheless, through nonspecific interactions, result in binding of some of the phage to the solid support. Alternatively, or in addition, the library may contain members which actually bind quite strongly to immobilized target, in a specific manner, but fail to bind to the target when the target is dissolved in solution. When these false positive binders are eluted and amplified, effort is wasted in obtaining characterization of proteins which, when later tested under other conditions (for example, with the target in solution) prove to be other than those desired.

Still another problem relates to lack of specificity for the target. Phage-displayed proteins may be retained, legitimately enough because the interaction with the target is real, but where the protein lacks sufficient specificity to serve the purpose for which it is intended. Another consequence of this problem arises in cloning families of proteins, such as kinases, where known kinase inhibitors are coupled to solid supports to retrieve such kinases from phage-display libraries. As the specificities for kinases overlap, the same kinase may be repeatedly isolated even though different inhibitors are used as bait coupled to solid support at the expense of additional kinases that uniquely interact with a particular inhibitor or subset of inhibitors. The specificity filter aspect of the invention addresses this problem.

The Affinity Filter

These problems can be avoided using the pre-validation and affinity filter system of the invention. Rather than simply contacting the phage display library with the solid support to which target is bound, the library/solid support interaction is also tested under conditions where the solution containing the phage display library includes various concentrations of the target in soluble form. This target in solution then competes with the immobilized target for the phage-displayed protein in those instances where the protein actually binds the solubilized target. Thus, there are three types of results obtained depending on whether the displayed protein is (1) a false positive where the protein creates the artifact of binding to the target when it is perhaps distorted by being immobilized, but does not bind the target in solution, (2) a protein with a high affinity for a target in solution (as well as a target in immobilized form) and (3) a protein with moderate affinity for the target in solution (as well as immobilized target).

When there is no competitive target in solution, both nonspecific or false positive proteins will be recovered along with high affinity interacting proteins; moderate binders may not be recovered, especially after multiple rounds of selection. As the concentration of dissolved target is increased, false positives will continue to bind substantially at the same level to the solid support at both low and high concentrations since these proteins do not bind to the dissolved target anyway. Thus, a phage-displayed protein that is recovered from the solid support both without the presence of dissolved target, and at high concentrations of dissolved target will be identified as a false positive.

High affinity binders are also readily identified. At low concentrations of the dissolved target, the proteins with high affinity interaction will no longer be detectably bound to solid support, since even a low concentration of the target can bind with sufficient affinity to diminish the protein available for binding to the support. Thus, an apparent binder which can no longer be recovered from the solid support in the presence of low concentrations of dissolved target is identified as a high affinity binder.

Moderate binders can also be identified by this method. While high concentrations of dissolved target can successfully compete with the immobilized form, a low concentration will not be sufficient to couple enough moderate affinity protein to prevent its binding to the support. While the moderate affinity protein was also bound in the absence of the soluble target, its presence may have been undetectable by virtue of having been “swamped” by the high affinity binding protein as described above.

At high concentrations of soluble target, both proteins which bind strongly and those which bind moderately to dissolved target are successfully competed away from binding to the column, and only the false positives remain bound.

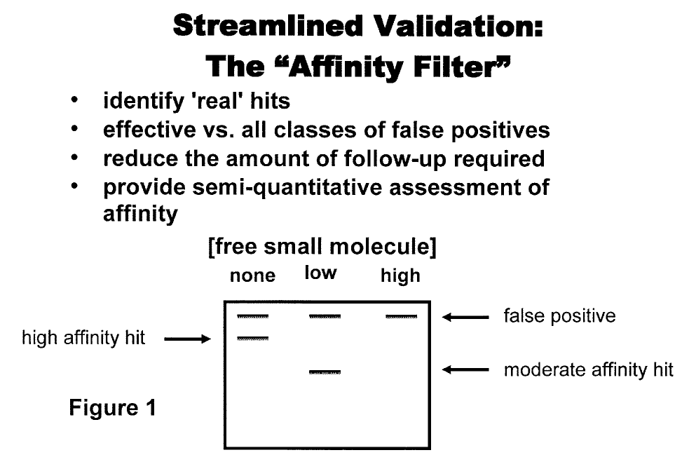

A summary of the foregoing discussion is found in FIG. 1. These results are shown as hypothetical separation gel of either proteins or the corresponding nucleic acids associated with amplified phage which is recovered from target immobilized to solid support. As shown, in the absence of soluble target (lane 1), both high affinity and nonspecific proteins are recovered from the column. At low concentrations of target (lane 2), nonspecific binders still are retained but the high affinity protein is no longer recovered; instead a moderate affinity protein is obtained. At high concentrations of target (lane 3), only nonspecific binders remain.

In more detail, FIG. 1 shows the results of a model selection experiment to illustrate the benefits of the affinity filter. The results of three selection experiments are shown in lanes 1, 2 and 3. The same immobilized small molecule bait and T7 cDNA library is used in all three selections; however, in lanes 2 and 3, free cognate competitor small molecule is included during binding at low or high concentration, respectively. Lanes 2 and 3 thus represent the affinity filter. Three phage clones were selected in these experiments. The first clone appears in all three selections is not eliminated at even the highest competitor concentration. This clone is thus a false positive. The second clone appears only in the selection that lacks competitor and is thus a high affinity true positive. The third clone appears only in the low competitor selection and is thus a low affinity true positive. In the absence of the affinity filter (lane 1), the low affinity true positive clone is lost since the high affinity true positive takes over the selection; however, with the low concentration affinity filter, which selectively removes the high affinity binder, the low affinity binder is able to emerge.

This technique has been successfully applied to suppress the enrichment of a high affinity methotrexate binding protein (DHFR), and permitted selection of a low affinity methotrexate binder (KIAA0663) that was not enriched in the absence of the affinity filter, as shown in Example 5.

Thus, to identify a false positive, it is theoretically necessary only to ascertain that the phage-displayed protein is retained by the solid support containing immobilized target at high concentrations of target in solution for competition. However, because the occurrence of such binding is problematic only in the case of attempts to find proteins that actually do bind to target, generally, data will also be provided under conditions where either target is not present in solution or present only in low concentrations.

Phage-displayed proteins with a high affinity for the target in solution can be identified by comparing retention to the solid support in the presence of low concentrations of competing target in comparison to retention in the absence of competing target. Thus, only two determinations are required.

For moderate affinity binders, although theoretically, these may be retained under some circumstances when dissolved target is absent, as a practical matter, if high affinity binding proteins are present in the phage display library, any binding may go undetected as overwhelmed by the competition from the high affinity binders. However, even though retention in the absence of dissolved target may be low or undetectable, retention should be readily detected in the presence of low concentrations of dissolved target.

As to the quantitation of “low” and “high” concentrations of dissolved target, the numerical value of these concentrations will be dependent on the actual values of high and low affinity binding in the context in which the phage display screening takes place. For example, as described in WO01/8234, the target may be a small molecule drug where the goal is to ascertain the biological receptor with which the drug interacts. Presumably, this receptor will have a higher affinity for the drug than alternative receptors present in the organism to which binding is more moderate, but wherein binding may result in side effects of the medication. Since the receptor for the drug in unknown, so too is the value of the dissociation constant which describes the interaction between the drug and its receptor. Thus, the levels of concentrations defined as “high” or “low” must be defined empirically. A “low” concentration might arbitrarily, then, be defined as 1-10 nM; if this concentration fails to disrupt the retention of the receptor from the immobilized drug target, the concentration would be increased to, for example, 10-20 nM, and thus incrementally to 20-50 nM, 50-100 nM, 100 nM-1 μM, 1 μM-10 μM and so on. The appropriate concentration would be identified as that which results in substantial lack of retention of the phage-displayed protein “hit.” A “low” concentration would then be selected from the ranges below that which was selected as “high.” Preferably a range at least 10-100 fold lower would be selected to identify moderate binders.

Alternatively, the goal may simply be to find a phage-displayed protein which binds with a predetermined affinity for the target. In this case, a “low” concentration of dissolved target would be a concentration which is at least equal to, and preferably higher than, the value of the dissociation constant describing the desired affinity. Thus, if the dissociation constant predicts an IC₅₀ of 0.1 μM, a “low” concentration would desirably be 0.5-1 μM. “High” concentration would be one or two orders of magnitude higher than that determined as “lower.”

In addition to permitting qualitative validation of specific protein/target interactions and exposing moderate-strength interactions of this type, the methods of the invention permit an estimate or, indeed, a quantitative determination of the dissociation constant between the target and a displayed protein. A rough estimate can be obtained by determining the minimum concentration of soluble target required to effect disappearance of the protein from binding to solid support. For example, if the protein under consideration appears no longer to be bound to the support at a concentration of 1 μM, this suggests that the K_{d} is less than, or equal to, that amount. If a 10 μM concentration is required, but the protein is still bound at a soluble target concentration of 1 μM, the K_{d} is putatively less than 10 μM but more than 1 μM.

In more detail, the K_{d} value is the concentration of competitor that reduces phage binding to the target retained on a solid support by 50% relative to a control where there is no competitor in solution. Typically, in the absence of competitor, only a small proportion of the phage are actually bound. Frequently, only 0.1%-10% of the phage bind to solid support in the absence of competitor; thus, for example, if only 1% of the phage bind to the retained target in the absence of competitor, the K_{d} will be determined as the competitor concentration that reduces the fraction bound from 1% to 0.5%.

A quantitative determination of K_{d} can be obtained by plotting the fraction of the protein bound to the solid support at varying concentrations of soluble target. The concentration at which half of the protein is bound and half unbound as compared to control when no competition is present thus represents the K_{d} for dissociation between the protein and target.

Thus, the invention method provides a number of advantages: first, it eliminates the necessity to expend time and resources in characterizing what may turn out to be a false positive interaction between a protein and a selected target; second, it exposes moderate affinity binders; and third, it permits calculation of affinity constants for specific interactions.

Detailed Description of Illustrative Selection Protocol with Affinity Filter

For a first round of selection, a cleared lysate containing the phage library is prepared by infecting log phase (A₆₀₀˜0.7) E. Coli BLT 5615 cells grown in 2×YT medium with a T7 phage library (M.O.I.˜0.05). The infected cells are shaken at 325 rpm at 37° C. until the lysate has cleared. The lysate is then aliquotted into 2 ml flip top tubes and spun in a microfuge at full speed for 10 minutes. The cleared supernatant is removed and used in the first round of selection in the form of a “lysate cocktail.” The final “lysate cocktail” solution to be tested contains 0.645× cleared lysate, 0.2× Seablock blocking agent buffer (Pierce #37527 Seablock/1% BSA/0.05% Tween 20, abbreviated SBTB); 1% BSA; 0.5% TritonX-100; and 0.05% Tween 20.

In the meantime, polystyrene plates which will contain immobilized bait are prepared as follows. Typically, four plates (3 polystyrene flat bottomed; 1 polypropylene round bottomed) are prepared. These plates are blocked with 200 ml SBTB per well.

Dynabeads M280 (Streptavidin (Dynal #602.10)) are resuspended by shaking and swirling; the beads are suspended at 10 mg/ml, as described in the next paragraph, and 0.4 mg (40 ml of the stock) are used per assay well.

The beads are washed 3 times and resuspended in 1×PBS/0.05% Tween 20 (PBST) to 10 mg/ml and distributed to 2 ml tubes—i.e., 1 tube per bait being tested. The biotinylated bait is added to the tubes at a molar ratio of 1:1 (bait:biotin-binding capacity), mixed and incubated on the rotator for 30 min at room temperature. Biotin is then added to all tubes at a molar ratio of 2:1 (biotin:biotin-binding capacity) and the tubes are incubated for another 30 min on the rotator.

The polystyrene plates prepared above, without removal of SBTB, are then supplied with the beads at 40 μl of beads per well. Four wells will be used for each bait:lysate-cocktail pair—two “selection” wells and two “affinity filter” wells. The plates containing the beads are shaken briefly at 700 rpm (wash 1), followed by pelleting, decanting, and another wash with SBTB (wash 2), followed by a third wash where the beads are shaken for >15 min. in SBTB.

200 μl of the lysate cocktail is added to the selection wells and 190 μl of the lysate cocktail and 10 μl of an affinity filter stock are added to the affinity filter wells. The affinity filter is prepared as a 20× concentrated stock of dissolved bait in DMSO. Plates containing blocked beads and either the lysate cocktail alone or the lysate cocktail with competitor are shaken at 700 rpm for 1 hour at room temperature.

The reactions are then transferred to a fresh blocked 96-well polystyrene plate. The beads are pelleted, decanted, and 150 μl of SBTB/0.5% Triton X-100 (SBTBT) is added to re-suspend the beads by shaking at 700 rpm for 5-10 seconds. The beads are washed three more times with 150 μl of SBTBT. On the fourth wash, the beads are transferred to a fresh blocked polystyrene 96-well plate.

The beads are then eluted by re-suspending in 200 μl of PBST containing 2 μM of dissolved bait and shaking at 700 rpm at room temperature for 30 minutes. The beads are pelleted and the eluate is removed.

The eluates are then analyzed as described below.

For additional rounds of selection, log phase cells are dispensed at 1 ml/well in a 96-well, 2 ml deep, well block. These cells are infected with 100 μl of eluate from the previous round and covered with AirPore sealing tape, and shaken at 325 rpm on the slant-rack at 37° C. Once lysis is complete, the block is chilled on ice for 5 min., and centrifuged 15 min. in the Qiagen centrifuge at top speed. The cleared lysates thus obtained are diluted 1:100 with 2×YT and lysate cocktails prepared by adding 71 μl of the components described above SBTB, BSA, TritonX, Tween 20 to blocked polypropylene 96-well plates and 129 μl of the cleared diluted lysates added. The bait bound to beads is then added and the bound phage eluted as described above.

Analysis of Eluates for Selection Protocol

For analysis, 40 cycles of PCR are performed on 3.5 μl of eluent in 25 μl reaction using primers T7 Up and T7 Down and Qiagen Taq polymerase. The primers bracket the phage inserts and are common to all phage in the library. Insert size varies greatly in a typical library so that when products from a crude lysate are separated on agarose gel, a smear is obtained. Typically, 10 μl of the PCR reactions are run on 2% agarose alongside a 100 bp ladder. Success in selection is shown by obtaining discreet bands. The number and relatively intensity of the discreet bands is indicative of the diversity of the selected population.

Forward Screening

The general principles applied in the affinity filter aspect of the invention above can also be advantageously employed in a “forward screen” to find alternatives to a target molecule for which an interaction is known or discovered with a phage-displayed protein or peptide. In this approach, large numbers of alternative candidate molecules can be screened rapidly to identify those which will also bind the phage-displayed proteins. The affinity with which the alternative, competitor molecule binds the protein can also be preselected by adjusting the concentration of candidate. If higher affinity is desired, lower concentrations of the candidate are offered and success in dislodging phage from immobilized parental molecule is required at these lower concentrations.

As used herein, “parental molecule” refers to a target molecule which has been identified or is known to bind to a particular phage-displayed protein or peptide (“peptide” and “protein” are used interchangeably herein). This parental molecule is immobilized to solid support using any conventional method as described above. The solid support can take any convenient form such as beads, surfaces, microtubes, and the like. The immobilized parental molecule is contacted with a phage lysate where the lysate contains not a library, but a single phage clone displaying a protein to which the parental molecule is known to bind. This interaction is tested in a sample which contains at least one competitor molecule and a sample which contains no competitor. The phage is eluted from the solid support in each case and the titers compared in the presence and absence of the candidate molecule. Successfully binding candidates will lower the titer as compared to the titer obtained when the competitor is absent.

This approach offers the ability to screen large numbers of candidate molecules rapidly by conducting the initial competition reactions supplying the candidate molecules in pools. The number of candidates in each pool is arbitrary but may be 2, 5, 10, 50, or even more. If the pool is unsuccessful in lowering the titer of bound phage, no member of the pool need further be tested. If the pool is successful, individual candidates can be tested, or intermediate size pools of those originally used can be employed. For example, if the initial pool contains 50 candidates, the testing can be continued with 5 pools each containing 10 of the 50 candidates. Only successful pools are then further subdivided for subsequent rounds of testing.

The results with a single candidate also permit an estimation of the dissociation constant of the candidate. The lower the concentration of the candidate required to lower the titer, the higher the affinity of the candidate for the displayed protein. Under the conditions of the assay, to select molecules with a K_{d} 1 μM or below, each competitor molecule would be present in the assay at 10 μM; a 1 μM binder would reduce phage binding to the parental molecule by a factor of 10. When higher affinity binding is sought, the competitor concentration is reduced to lower values.

The conditions of the assay are important in order to provide the correct quantitative results. One might assume that the concentration of competitor required to dislodge a fixed proportion of the phage would be dependent on the value of the K_{d} for the parental molecule as well. Also, in a large excess of phage-displayed protein, the competitor would not necessarily displace phage already bound to parental molecule, but rather could bind to the excess phage.

Thus, the assay is run based on certain assumptions wherein it can be shown that the concentration of competitor that reduces the binding to the immobilized parental molecules by 50% is equal to the K_{d} for the competitor.

These assumptions and conditions are as follows:

First, the concentration of the phage-displayed protein must be less than the K_{d} for the competitor. Second, the concentration of the immobilized parental molecule must be less than the K_{d} for the immobilized parental molecule.

It is straightforward to provide conditions for the assay wherein these assumptions are met. The concentration of phage-displayed protein in the assay is kept quite low, typically less than 20 nM; when very tight binders are sought, the phage is diluted to a lower concentration. Thus, there is no excess of phage-displayed protein.

The apparent K_{d} for the competitor molecule will depend on the K_{d} for the immobilized parental molecule only when the concentration of immobilized parental molecule is greater then its own K_{d}. Thus, in the assays of the invention, typically, the concentration of immobilized parental molecule ranges from 100 nM-1000 nM which is generally in the range of K_{d}'s for the immobilized parental molecules. If there is any doubt that the concentration of the immobilized parental molecule is in fact less than its K_{d}, the competition can be performed at two concentrations of the immobilized parental molecule to ensure consistency. It is particularly important to test these assumption when high affinity competitors are sought.

When these assumptions are valid, competitive binding can be described by the following equation:

f/f₀=K_{comp}/(K_{comp}+[comp])

where f is the fraction of phage bound to the immobilized molecule in the presence of dissolved competitor; f₀ is the fraction bound in the absence of dissolved competitor; K_{comp} is the equilibrium dissociation constant (K_{d}) for the interaction between the phage-displayed protein and the dissolved competitor; [comp] is the concentration of the dissolved competitor. At 50% competition, f/f₀=0.5, and K_{comp}=[comp].

If the foregoing assumptions are not valid, the apparent K_{d} for the competitor as determined by the assay will be overestimated—i.e. the binding to the phage is actually tighter than it appears from the assay. Again, if there is doubt, the assays can be run at more than one concentration of the immobilized parental molecule to ensure that the assumptions are met.

This approach to forward screening has several advantages. First, it employs the same general techniques as those of the affinity filter, thus permitting the discovery of alternative binders without the need for further assay development. The screened molecules do not need to be immobilized, and the assay is amenable to scale-up and is semi-quantitative. That affinity of the successful binders can be discerned from the assay itself.

Means are commercially available to verify the specificity of the binding of successful competitors. For example, Proteome Scan™ assays can be used to assess binding against a large number of other proteins, and those which bind nonspecifically can be discarded.

Detailed Description of Illustrative Protocol for Forward Screening

The detailed procedure is substantially equivalent to that set forth above for the selection protocol with affinity filter. A cleared lysate is prepared containing the phage displaying the protein against which a multiplicity of compounds are to be tested for binding. A single clone displaying this protein is substituted for the phage library in infecting log phase cells, typically E. coli BLT 5615. Otherwise, the cleared lysate is obtained as described above. The preparation of the polystyrene and polypropylene plates is identical to that in the selection procedure as is the preparation of the Dynabeads; however, the Dynabeads contain an immobilized form of the “parental” molecule which is known to bind the displayed protein. Competitors to be tested are added to the wells rather than varying concentrations of the immobilized molecule. Analysis is conducted by titration of the phage eluates, rather than size separation.

In more detail, a cleared lysate containing the phage clone that displays the protein for which binding partners are to be found is prepared by infecting log phase (A₆₀₀˜0.7) cells, typically E. coli BLT 5615 cells grown in 2×YT medium with the appropriate T7 phage clone (M.O.I.˜0.05). The infected cells are shaken at 325 rpm at 37° C. until the lysate has cleared. The lysate is then aliquotted into 2 ml flip top tubes and spun in a microfuge at full speed for 10 minutes. The cleared supernatant is removed and used in the form of a “lysate cocktail.” The final “lysate cocktail” solution to be tested contains 0.645× cleared lysate, 0.2× Seablock blocking agent buffer (Pierce #37527 Seablock/1% BSA/0.05% Tween 20, abbreviated SBTB); 1% BSA; 0.5% Triton X-100; and 0.05% Tween 20.

In the meantime, plates which will contain immobilized reaction mixtures are prepared as follows. Typically, four plates (3 polystyrene flat bottomed; 1 polypropylene round bottomed) are prepared. These plates are blocked with 200 ml SBTB per well.

Dynabeads M280 (Streptavidin (Dynal #602.10)) are resuspended by shaking and swirling; the beads are suspended at 10 mg/ml, as described in the next paragraph, and 0.4 mg (40 [symbol] of the stock) are used per assay well.

The beads are washed 3 times and resuspended in 1×PBS/0.05% Tween 20 (PBST) to 10 mg/ml and the biotinylated parental compound known binder is added at a molar ratio of 1:1 (bait:biotin-binding capacity), mixed and incubated on the rotator for 30 min at room temperature. Biotin is then added at a molar ratio of 2:1 (biotin:biotin-binding capacity) followed by incubation for another 30 min on the rotator.

The flat bottomed polystyrene plates prepared above, without removal of SBTB, are then supplied with the beads at 40 μl of beads per well. The compounds to be screened in the forward screen will typically be tested first in pools; components of successful pools can then be tested in the same manner separately or in smaller pools. For each pool to be tested, there are positive control wells which contain no pool of competitors, negative control wells which contain the Dynabeads bound only to biotin and test wells which contain the pools of competitors. The plates containing the beads are shaken briefly at 700 rpm (wash 1), followed by pelleting, decanting, and another wash with SBTB (wash 2), followed by a third wash where the beads are shaken for >15 min. in SBTB.

The competitor pools are prepared as 20× concentrated stocks in DMSO; 10 μl of the 20× competitor pools and 190 μl of the lysate cocktail are added to the test wells.

200 μl of the lysate cocktail is added to the positive and negative control wells. The plates are then shaken at 700 rpm at 1 hour at room temperature.

The reactions are then transferred to a fresh blocked 96-well polystyrene plate. The beads are pelleted, decanted, and 150 μl of SBTB/0.5% Triton X-100 (SBTBT) is added to re-suspend the beads by shaking at 700 rpm for 5-10 seconds. The beads are washed three more times with 150 μl of SBTBT. On the fourth wash, the beads are transferred to a fresh blocked polystyrene 96-well plate.

The beads are then eluted by re-suspending in 200 μl of PBST containing 2 μM of parental known binder in solution and shaking at 700 rpm at room temperature for 30 minutes. The beads are pelleted and the eluate is removed.

The eluates are then analyzed by titration of the phage. The phage titer in the negative control should be 2-3 orders of magnitude lower than the positive control and highest in the positive control wells. A “fold competition” can be calculated by dividing the phage titer in the positive control by the phage titer in a competition well.

Specificity Filter

It may be desirable to obtain phage-displayed proteins that are specific for a particular compound, but which fail to bind certain additional compounds. As described above with respect to the forward screen, under certain conditions, a sufficient concentration of the compound with which interaction is not desired can be supplied so as to reduce the fraction of phage displaying the protein bound to solid support to any desired level. Thus, in this application, rather than assessing the ability of an additional compound to compete for the displayed protein with the immobilized target, the compound to which binding is not desirable can be added at sufficient concentration to prevent the coupling of the phage-displayed protein to the solid support containing the desired target. In this aspect, proteins can be identified that have desired specificity for a particular target compound, but fail to bind to other compounds which are thus used to filter out non-specific members of a phage display library.

In more detail, a first compound for which specifically binding proteins are desired is coupled to solid support and treated with a fluid containing a phage display library. The fluid also contains at least a second compound or a multiplicity of such second compounds for which binding is not desired. This second compound (or multiplicity of second compounds) will compete successfully for any proteins that interact with them, thus providing a binding advantage to phage-displayed proteins that are specific to the immobilized target. The method has wide application with respect to a multiplicity of analogous compounds that may interact with phage-displayed protein. In preferred embodiments, the compounds are other than proteins or peptides and are preferably commonly understood as “small molecules”—i.e., compounds with molecular weights less than 2,000 kD, preferably less than 1,000 kD, and more preferably less than 500 kD. These “small molecules” do not include peptides.

This aspect is particularly useful in cloning related proteins with overlapping specificities. Once a member of a class of proteins is cloned and recombinantly produced, its reactivity with a multiplicity of compounds can be assessed. Then a single compound for which additional members of the group might have affinity is employed as the target immobilized on a support and the remaining compounds that the retrieved protein binds are used as competitors. This permits recovery of additional members of the class that bind to the target compound but not to the remaining compounds that bind to the first protein recovered.

In the absence of this technique, the initially recovered member of class may continue to be recovered again and again, masking the presence of alternative members of the class with different specificity profiles.

Detailed Description of Protocols for the Specificity Filter

The detailed procedure is again substantially equivalent set forth above for the selection protocol with the affinity filter. A cleared lysate containing the phage library is prepared as described above. The details are identical to those described under the heading “Detailed Description of Illustrative Selection Protocol with Affinity Filter.” The preparation of immobilized compound and addition of alternative compounds to the lysates is as described, with the exception that rather than including a defined concentration of the immobilized compound in the lysate, the lysate is modified to contain a sufficient concentration of alternative compounds to substantially prevent retention of a protein that is able to bind both the immobilized compound and the alternative compounds provided to the lysate. The required concentrations, depending on the level of specificity desired, are calculated as described with respect to the forward screen aspect of the invention set forth above. The retained protein displaying phage are then eluted from the solid support and assayed as desired. If performed in the context of retrieving nucleotide sequences encoding desired proteins, the nucleic acid inserts in the phage are sequenced and the amino acid sequence encoded deduced.

The following examples are intended to illustrate but not to limit the invention.

### EXAMPLE 1

### Effect of Rapamycin Concentration on Binding of FKBP Proteins

The immunosuppressant, FK506 which is related to the antibiotic rapamycin structurally and in terms of its binding capability, is known to bind FK binding proteins (FKbp) 4 and 2 and to bind more strongly to FK binding protein 1 (two versions). A human brain cDNA library was cloned into T7 phage purchased from Novagen. One of the two FKbp1 clones was inserted using standard protocols of the Gateway™ Cloning Technology distributed by Life Technologies. The phage library used in this example contained these FKbp's spiked at 1:105. That is, phage clones displaying these proteins were added to a high complexity cDNA library where, in this instance, 10⁵ identical copies of the FKbp phage clones were added to 1010 non-FKbp phage. The library was treated with solid support to which FK506 was bound. The column was washed and eluted with 2 μM rapamycin to recover bound phage-displayed proteins.

The solid support with immobilized FK506 was contacted with the library in the presence of rapamycin as a competitor to FK506 where the concentration of rapamycin is 0, 24 nM, 240 nM or 2,400 nM. At each concentration, two rounds of selection were performed. That is, after treating with the fluid phase containing phage plus the noted concentration of rapamycin, the columns were washed and then eluted with 2 μM rapamycin to recover bound phage. The recovered phage were amplified in E. coli BLT5615, again applied to the solid support in the presence of the same concentration of rapamycin as previously, and the bound phage again eluted.

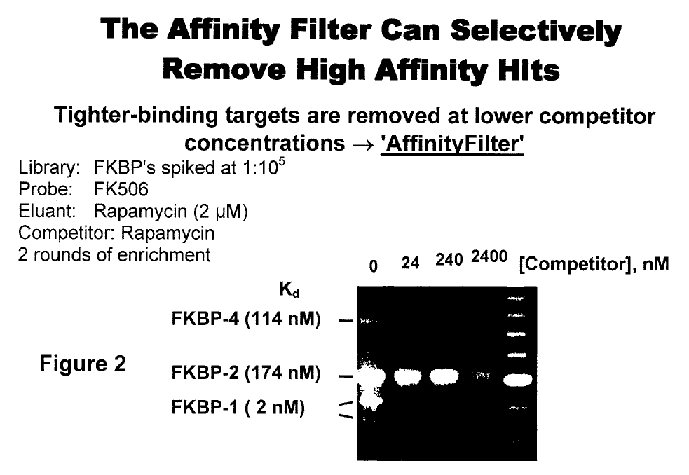

The phage eluted at each rapamycin concentration were PCR amplified and run on a gel. The results are shown in FIG. 2. As shown, FKbp1 is detected on the column only when no rapamycin is present (lane 1). FKbp1 was determined to have a K_{d} of 2 nM. However, the solid support is able to retain FKbp2 and FKbp4 at concentrations of 24 nM (lane 2) and 240 nM (lane 3), but no longer are present on the column at 2,400 nM (lane 4). These proteins were determined to have K_{d}'s of 114 nM and 174 nM respectively.

The FKbp1 clones, which have the highest affinity for rapamycin (K_{d}=2 nM), are eliminated at the lowest affinity filter concentration (24 nM, lane 2). By contrast, FKbp's −2 and −4, which have lower affinities for rapamycin (K_{d}=174 nM and 114 nM, respectively), are only eliminated at the highest affinity filter concentration (2400 nM). The data are semi-quantitative and accurately predict that the rapamycin-FKbp1 K_{d} is <24 nM and the rapamycin-FKbp2/4 K_{d}'s are <2400 nM.

### EXAMPLE 2

### Affinity Filter for trkA-PY490 Phosphopeptide

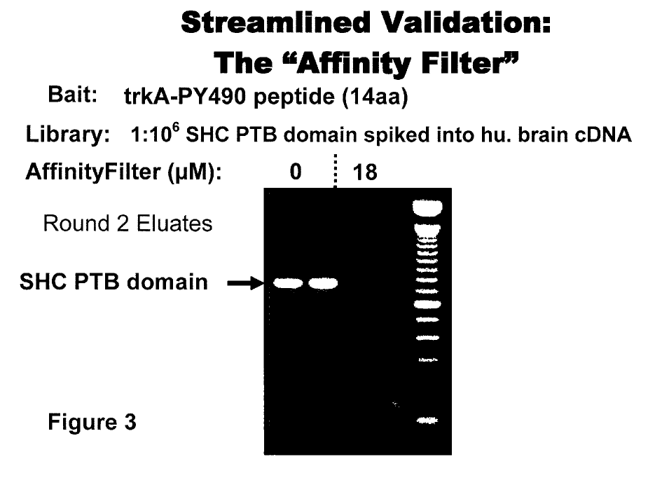

In a manner similar to that set forth in Example 1, trkA-PY490 phosphopeptide was used as bait to select for phage displaying the SHC PTB domain. Phage displaying the SHC PTB domain were “spiked” into a human brain T7 phage cDNA library at a level of 1:106. The results are shown in FIG. 3. Duplicate selections were set up in the absence (lanes 1 and 2) or the presence (lanes 3 and 4) of an affinity filter (18 μM free trkA-PY490 peptide). The data after two rounds of selection illustrate that a single phage clone is strongly enriched only in the selections that lacked the affinity filter (lanes 1 and 2). Hence, this clone, which indeed displays the SHC PTB domain, is a validated binder since it was eliminated in the affinity filtered selections.

### EXAMPLE 3

### Determination of K_{d}

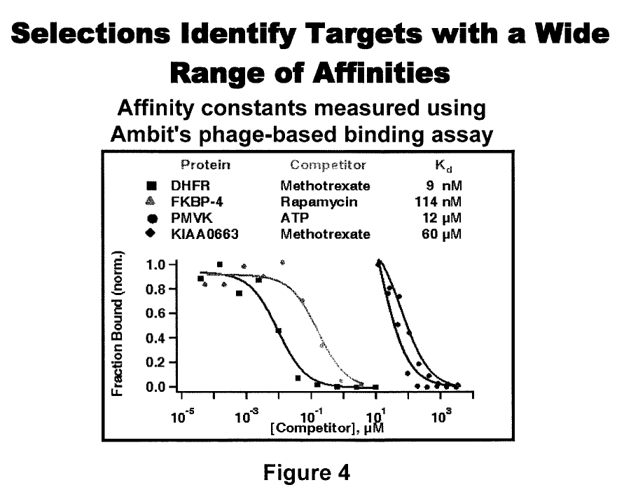

In a manner similar to that set forth in Example 1, known protein/target interactions were used in a series of experiments in which the concentration of competitor was varied over several orders of magnitude and the fraction of the phage bound to solid support as compared to control was determined. As shown in FIG. 4, the strongly interacting pair methotrexate and DHFR give results whereby the DHFR is detectably bound to the solid support only in the range of 0.1-1 nM; at concentrations of soluble methotrexate of just over 10 nM, the DHFR is no longer bound to the support. The y axis of the graph has been normalized so that the binding which occurs in the absence of competitor is designated 1.0. However, the actual range for non-normalized data is 0.1% to 0.001%. A calculated value where 50% of the DHFR is bound to the column compared to control provides a K_{d} of 9 nM. In comparison, the weak interaction between methotrexate and KIAA0663 shows 50% of control binding only at about 100 μM. The plotted results in FIG. 4 also confirm 50% of control binding for displayed FKbp4 at approximately 100 nM rapamycin. Displayed PMVK is 50% bound to immobilized ATP as compared to control when the concentration approximates the K_{d} of 12 μM.

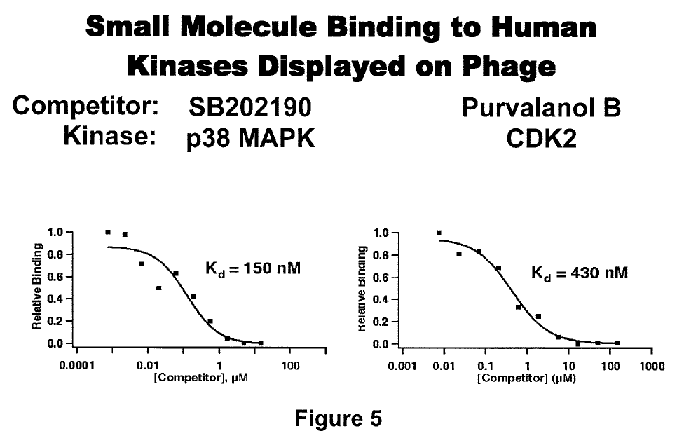

As another example, as shown in FIG. 5, known kinase inhibitors have been used as immobilized bait to bind their cognate phage-displayed kinases. In the left panel, the p38 MAP kinase was bound to immobilized SB202190 in the presence of various concentrations of free, unlinked SB202190. K_{d}=150 nM was measured for the interaction between the displayed p38 MAP kinase and the free, unlinked SB202190 molecule. In the right panel, the CDK2 kinase was bound to immobilized purvalanol B in the presence of various concentrations of free, unlinked purvalanol. K_{d}=430 nM was measured for the interaction between the displayed CDK2 kinase and the free, unlinked purvalanol molecule.

### EXAMPLE 4

### Application of a Forward Screen Procedure

Using the detailed procedure set forth hereinabove, p38 kinase displayed on phage was bound to immobilized SB202190 as described in Example 3. Various competitors were tested at 1 μM and 10 μM concentrations to assess whether displacement of phage from the immobilized support could be detected. As shown in Table 1, the experimental observations were consistent with expectations when compounds known to interact with the kinase were used as competitors and when compounds known not to interact with the kinase were used as competitors.

TABLE 1
Forward Screens: p38/SB202190

| Competitor (1 and 10 μM) | Exp. | Obs. |
|---|---|---|
| ATP (10 and 100 μM) | − | − |
| AMP-PNP | − | − |
| Bisindoylmaleimide | − | − |
| Bisindoylmaleimide-dimethyl | − | − |
| CDK2 inhibitor (oxindole) | − | − |
| Purvalanol A | − | − |
| Purvalanol B | − | − |
| Staurosporin | − | − |
| PD98059 | − | − |
| SB202474 | − | − |
| PD169316 | + | + |
| SB202190 | + | + |
| SB202190-OMe | + | + |
| SB203580 | + | + |
| SB203580-iodo | + | + |
| SB220025 | + | + |
| ZM336372 | + | + |
| p38 inhibitor (Calbio.) | + | + |

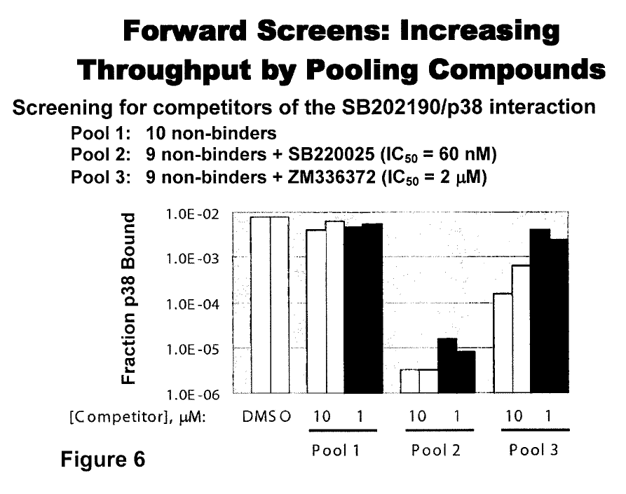

Based on this information, it was demonstrated that the throughput of the assay can be enhanced by pooling compounds. The results are shown in FIG. 6.

When DMSO was used as a negative control, the fraction of p38 bound to immobilized SB202190 was roughly 10⁻². When a pool of 10 compounds known to be non-binders was substituted for DMSO, either at 10 μM of each compound or 1 μM of each, little diminution in binding occurred. A second pool which contained nine non-binders and a strong binding compound, SB220025 (with an IC₅₀ of 60 nM) was then substituted for DMSO. When these compounds were present either at 10 μM or 1 μM, the fraction of p38 phage bound was diminished to <10⁻⁵. When a similar pool was employed, but substituting the more weakly binding ZM336372 for the more tightly bound SB220025 (IC₅₀ of 2 μM) the fraction of p38 bound at a 110 μM concentration of pool compounds fell below 10⁻³ M; a less dramatic decrease was obtained when the pooled compounds were supplied at 1 μM.

### EXAMPLE 5

### Recovery of Moderate Binder

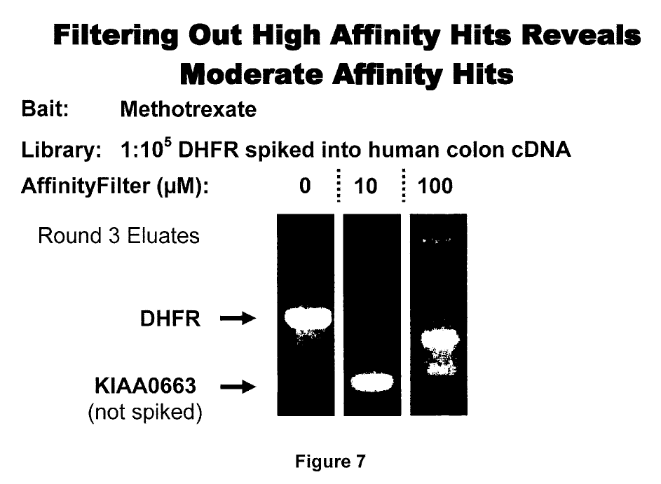

DHFR was spiked into a human colon phage cDNA library at a level of 1:105. The library was probed with immobilized methotrexate, generally as described. As shown in FIG. 7, after three rounds of selection in the absence of dissolved methotrexate, DHFR was the exclusive species isolated (lane 1).

In the presence of 10 μM methotrexate, however, the high affinity binder DHFR is no longer apparent, and a new clone (KIAA0663) predominates that was not observed in the absence of dissolved methotrexate (lane 2).

At the higher concentration of 100 μM methotrexate, neither KIAA0663 nor DHFR is present (lane 3), indicating that both are true positives.

These data are semi-quantitative and predict that KIAA0663 is a low affinity binder and that DHFR is a high affinity binder. Detailed binding experiments have validated this prediction: the K_{d} for DHFR/methotrexate is 6 nM and the K_{d} for KIAA0663/methotrexate is 60 μM.

### EXAMPLE 6

### Selection of Compound-Specific Proteins Using the Specificity Filter

A library containing DNA inserts encoding fragments of 122 kinases was prepared in T7 phage as described hereinabove. Four different compounds, known to bind at least some kinases were immobilized on Dynabeads M280 streptavidin (Dynal No. 602.10)) as also described hereinabove. The phage lysates were treated with the derivatized beads in the presence of 10 μM of the immobilized compound (affinity filter) or 10 μM of the three alternative compounds (specificity filter). Thus, for example, when the immobilized compound was staurosporine, for the affinity filter, 10 μM staurosporine was included in the lysate; for the specificity filter, 10 μM each of SU5402, Purvalanol B, and SB202190 were included.

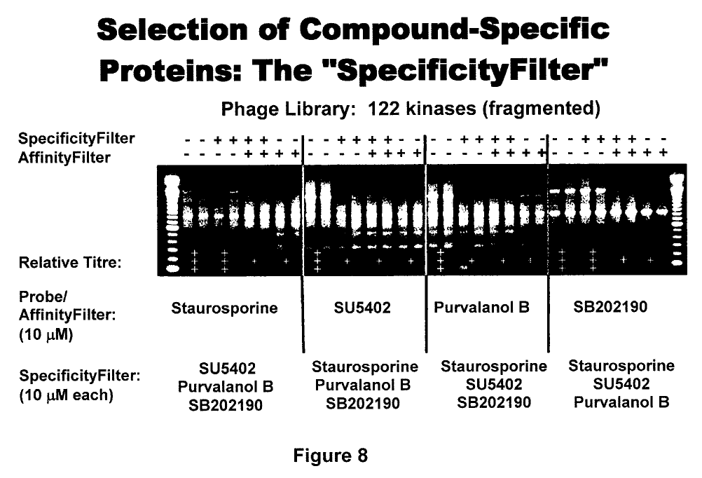

The results are shown in FIG. 8, which depicts gels displaying PCR amplified eluates of phage under the conditions described above. The presence of compounds constituting the specificity filter and/or affinity filter is noted at the top of each gel. In each case, the eluate was obtained from treating the immobilized compound with the lysate alone, with lysate containing the three alternative compounds; the lysate containing both the immobilized compound and the three alternative compounds, and the lysate containing 10 μM of the immobilized compound alone. Titers are indicated below the gels +++ representing high and + representing low titers.

As shown, in the presence of the affinity filter, the major component of all the eluates found in the absence of any free compound in the lysate disappears, as evidenced by the disappearance of bands on the gel and/or the titers indicated below the gels. When only the specificity filter is employed, in the case of staurosporine and SB202190, the results are similar to those obtained in the absence of the filter; however, for SU5402 and Purvalanol B, the major retained component is no longer present indicating that the originally bound protein binds to at least one of the alternative compounds as well as to the immobilized compound.

1. A method to identify a protein that binds to a first target compound but fails to bind to at least a second compound which method comprises
contacting a multiplicity of phage-displayed proteins with a solid support to which said first target compound has been immobilized in the presence of a sufficient concentration of said second compound to substantially prevent phage displaying proteins which bind both said first target compound and second compound from being retained on said solid support; and
recovering phage displaying protein from said solid support;
whereby said recovered phage-displayed protein is identified as binding specifically said first target compound, but not said second compound.

2. The method of claim 1, wherein said multiplicity of phage-displayed proteins are kinases.

3. The method of claim 1, wherein said fluid further contains, in addition to said second compound, additional compounds that are not target compounds.

4. The method of claim 1, wherein at least one of said first and second compound is other than a protein or peptide.

5. The method of claim 4, wherein at least one of said first and second compound is a small molecule.
